(19) United States
(12) Patent Application Publication
(10) Pub. No.: US 2024/0240105 A1
(43) Pub. Date: Jul. 18, 2024

(54) IMMOBILIZED ENZYME FIBER REACTOR AND OIL REFINING USING THE SAME

(71) Applicant: CHEMTOR, LP, Lockhart, TX (US)

(72) Inventors: Leslie WOOD, San Marcos, TX (US); Scott D. KOHL, Maize, KS (US)

(21) Appl. No.: 18/420,879

(22) Filed: Jan. 24, 2024

Related U.S. Application Data
(63) Continuation of application No. PCT/US2024/011361, filed on Jan. 12, 2024.
(60) Provisional application No. 63/499,063, filed on Apr. 28, 2023.
(60) Provisional application No. 63/479,682, filed on Jan. 12, 2023.

(51) Int. Cl. C11B 3/00; C12M 1/02; C12M 1/40; C12N 9/16; C12N 11/04; C12N 11/082; C12N 11/091

(52) U.S. Cl. CPC C11B 3/003; C12M 21/18; C12M 27/00; C12N 9/16; C12N 11/04; C12N 11/082; C12N 11/091; C12Y 301/01004; C12Y 301/01032; C12Y 301/04003

(57) ABSTRACT

An immobilized enzyme fiber reactor includes a plurality a fibers disposed within a hollow conduit. The fibers have an enzyme, such as a phospholipase, attached thereto. The enzymes can be attached to the fibers via an anchor group and, optionally, a bifunctional crosslinker. The enzymes can be applied, stripped, and reapplied without disassembling the reactor or discarding the fibers. The immobilized enzyme fiber reactor can be used to treat oils including phospholipids, such as soybean oil, and reduce an impurity content thereof.

### CROSS-REFERENCE TO RELATED APPLICATIONS

The present application is a continuation of PCT International Patent Application No. PCT/US2024/011361 filed Jan. 12, 2024, entitled “Immobilized Enzyme Fiber Reactor and Oil Refining Using the Same,” which claims benefit of priority to U.S. Provisional Patent Application No. 63/479,682 filed Jan. 12, 2023, entitled “Immobilized Enzyme Fiber Reactor and Oil Refining Using the Same,” and U.S. Provisional Patent Application No. 63/499,063 filed Apr. 28, 2023, entitled “Immobilized Enzyme Fiber Reactor and Oil Refining Using the Same,” the disclosures of each of which are hereby incorporated by reference in their entireties.

### FIELD OF THE DISCLOSURE

The present disclosure relates to enzymatic processes facilitated by a fiber reactor. More particularly, the present specification is directed to an immobilized enzyme fiber reactor and the use of the same for refining oils.

### BACKGROUND

Vegetable, seed oils, waste oils, and animal fats and lipids (biologically derived lipids) such as distillers corn oil (DCO) and soybean oil (SBO) may contain undesirable components and impurities. In particular, SBO contains high levels of phospholipids and associated metal impurities. Phospholipids and metals can produce negative effects on the flavor, shelf-life, and color of the oil when used for food production or in formulated food products. When SBO is destined for renewable fuels via hydrogenation catalytic processing, the phospholipids and metals must be reduced to low levels so that the catalyst bed which drives hydrocarbon cracking is not poisoned by these impurities. The tolerable impurity level for renewable oils feedstocks is typically <3 ppm residual phosphorus and <10 ppm total metals, but impurity upper limit values specified by catalytic vendors continues to be reduced over time suggesting even lower levels of contaminants are important.

Traditionally, phospholipid removal or “degumming” is performed using a water wash containing citric or phosphoric acid wherein the hydratable phospholipids are removed by dissolving in the added water phase and separating the oil from the water by density differential. The non-hydratable phospholipids are made hydratable by pushing a proton onto the phosphate group as well as a chelation effect of acids, such as citric acid or phosphoric acid, with the multivalent metal ions, such as Ca²⁺ and Mg²⁺, which are associated with the phospholipids (see O'Brien, R. D. (2008). Soybean oil purification. Soybeans (pp. 377-408). AOCS Press). Water added during the degumming process is typically 2-3% relative to the mass of oil treated, the temperature is 50-80° C., and stir times are around 15 to 30 minutes with a short high shear mixing component. The treated oil is then centrifuged to separate the water from the oil. Typical values attained using this method yield residual phospholipids between 15-30 ppm and residual metals between 10-20 ppm which do not meet the specifications previously noted (see Jiang, X., Chang, M., Wang, X., Jin, Q., & Wang, X. (2014). A comparative study of phospholipase A1 and phospholipase C on soybean oil degumming. Journal of the American Oil Chemists' Society, 91(12), 2125-2134).

The remaining phospholipids are commonly removed via caustic refining. This process adds an alkali material, such as aqueous sodium hydroxide, to continue the refining process. As with the initial water-plus-acid steps mentioned above, the caustic process is performed at elevated temperatures with high shear mixing devices. After sufficient mass transfer and/or contact time, the alkali solution is removed via centrifugation. See FIGS. 1A-1C for reference (from Erickson, D. R. (1995). Neutralization. Practical handbook of soybean processing and utilization (pp. 184-202). AOCS Press). The method outlined in FIGS. 1A-1C produces low phosphorous oil which is also low in free fatty acid (FFA) concentration. Once-refined SBO using this method may have 0.035 to 0.060 wt % FFA, 0 to 1.1 ppm of phosphorous, 0 ppm of soap, and a peroxide value of 0.9 to 3.3 mEq/kg. In addition to the chemical, electrical, labor, and thermal operating costs, the capital costs for these steps are significant. The use of alkali ionizes free fatty acids causing them to wash into the spent water thereby removing those components and the concomitant mass from the resulting oil. For food applications, removal of FFA is desired, but for fuel applications, particularly renewable diesel and sustainable aviation fuel production, the FFA removal is unnecessary and represents yield loss from the oil available for conversion to fuel.

A more recent approach has included the addition of phospholipase enzymes such as PLA₁, PLA₂, and PLC types. PLA₁ and PLA₂ enzymes target the ester bonds at the sn-1 and sn-2 positions. PLC targets the sn-3 bond between the acylglycerol and phosphate groups (see FIG. 2 and Sampaio, K. A., Zyaykina, N., Wozniak, B., Tsukamoto, J., Greyt, W. D., & Stevens, C. V. (2015). Enzymatic degumming: Degumming efficiency versus yield increase. European Journal of Lipid Science and Technology, 117(1), 81-86; Majerus, P. W. (1992). Inositol phosphate biochemistry. Annual review of biochemistry, 61(1), 225-250; Murayama, K., Kano, K., Matsumoto, Y., & Sugimori, D. (2013). Crystal structure of phospholipase A1 from Streptomyces albidoflavus NA297. Journal of Structural Biology, 182(2), 192-196). As shown in FIG. 3, PLD activity cleaves the phosphate, releasing phosphatidic acid and an alcohol. FIG. 3 also shows the cleavage sites for other phospholipases discussed herein. Cleavage by PLA type phospholipase releases fatty acids from the phospholipid whereas PLC produces a diacylglycerol. The reaction mechanisms are shown in FIG. 4. In SBO, the most abundant phospholipids in the gums are phosphatidylcholine (PC) followed by phosphatidylethanolamine (PE), phosphatidylinositol (PI) and phosphatidic acid (PA). The structures of these compounds are shown in FIG. 5. PLA type enzymes will release free fatty acids (FFAs) which can either add to the oil content in a renewable fuels application or be saponified and washed away in an edible oil process. The PLC enzyme releases a diacylglycerol which increases the oil content and will not be washed away. Enzymatic digestion reduces oil losses compared to traditional refining.

The typical commercial procedure involving stirred enzymes includes water/citric acid treatment mentioned above, NaOH addition to bring the solution to the optimal pH of the enzyme, and addition of the enzyme dissolved in water followed by stirring for at least an hour. This process is summarized in FIG. 6 (see Yang, B., Zhou, R., Yang, J. G., Wang, Y. H., & Wang, W. F. (2008). Insight into the enzymatic degumming process of soybean oil. Journal of the American Oil Chemists' Society, 85(5), 421-425). With added enzyme, long stir times are necessary because the reaction between the enzyme and oil only occurs at the oil/water interface. Post enzymatic processing residual phospholipids are <10 ppm and metals are <5 ppm. While the addition of enzymes can produce cleaner oils, the long stir times with high shear and continual consumption of enzymes required to do so are not ideal due to capital, enzyme cost, and high energy requirements (see FIG. 7 showing reaction time and temperatures needed for enzymatic phosphorous removal from SBO).

### BRIEF DESCRIPTION OF THE DRAWINGS

Various embodiments of the present disclosure will be understood more fully from the detailed description given below and from the accompanying drawings. In the drawings, like reference numbers may indicate identical or functionally similar elements. Embodiments are described in detail hereinafter with reference to the accompanying figures, in which:

FIGS. 1A-1C are diagrammatic illustrations of a caustic refining process flow and a mechanism for the formation of non-hydratable phosphatides.

FIG. 2 illustrates hydrolysis cleaving mechanisms of the PLA₁, PLA₂, and PLC phospholipase enzymes.

FIG. 3 is an illustration of cleavage sites for PLA₁, PLA₂, PLC, and PLD phospholipase enzymes.

FIG. 4 illustrates hydrolysis cleaving mechanisms of the PLA₁, PLA₂, and PLC phospholipase enzymes.

FIG. 5 illustrates chemical formulas for phosphatidylcholine, phosphatidylinositol, phosphatidic acid, and phosphatidylethanolamine.

FIG. 6 is a diagrammatic illustration of a process flow of stirred enzymatic degumming.

FIG. 7 is graphs showing phosphorous reduction as a function of reaction stir time and reaction temperature for phospholipase PLA and PLC in a traditional stirred enzymatic reaction with SBO.

FIG. 8A is a partial cutaway diagrammatic illustration of a fiber reactor according to an embodiment of the present disclosure.

FIG. 8B is a diagrammatic illustration of mass transfer between immiscible phases within microchannels formed between the fiber of the fiber reactor of FIG. 8A.

FIG. 9 is a diagrammatic illustration of a fiber reactor according to an embodiment of the present disclosure.

FIG. 10 is a diagrammatic illustration of immobilized enzymes on a fiber substrate according to an embodiment of the present disclosure.

FIG. 11 is a graph showing phosphorous removal results of Example 2.

FIG. 12 is a graph showing magnesium removal results of Example 2.

FIG. 13 is a graph showing calcium removal results of Example 2.

FIG. 14 is a graph showing FFA levels measured in Example 3.

FIG. 15 is a graph showing FFA levels measured in Example 4.

FIG. 16 is a graph showing metal removal results of Example 8.

FIG. 17 is a graph showing FFA levels measured in Example 11.

FIG. 18 is a graph showing FFA levels measured in Example 12.

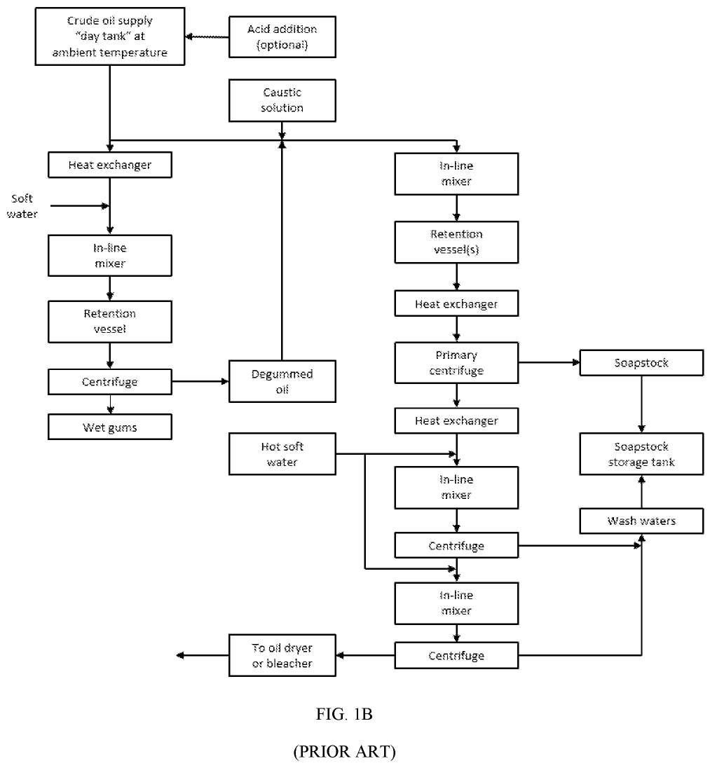

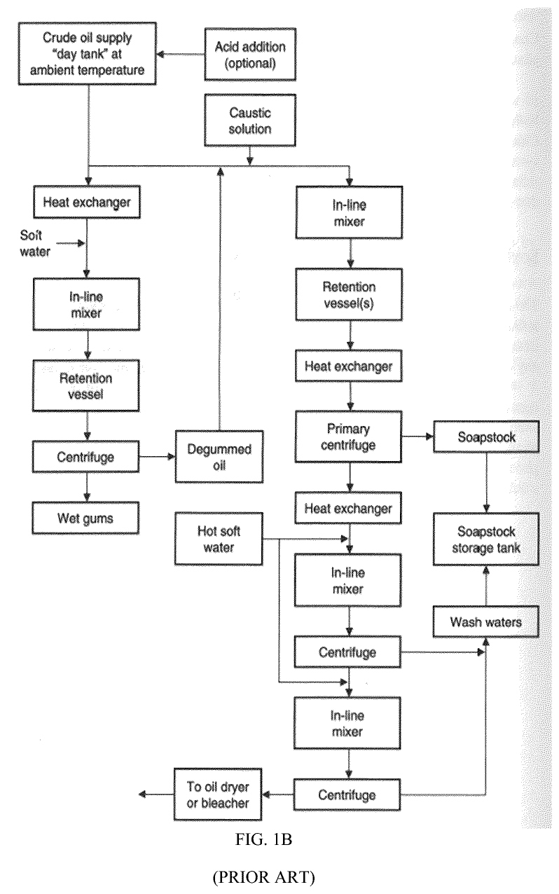

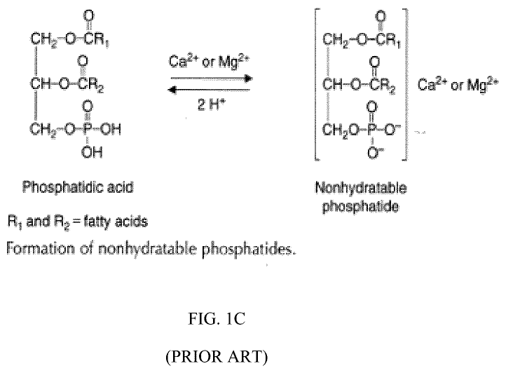

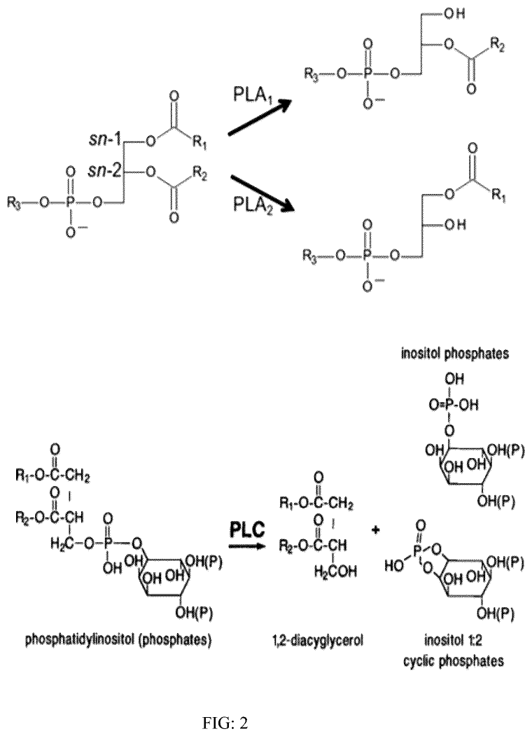

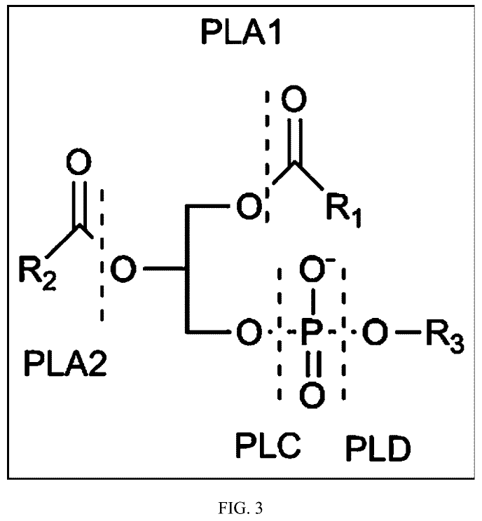

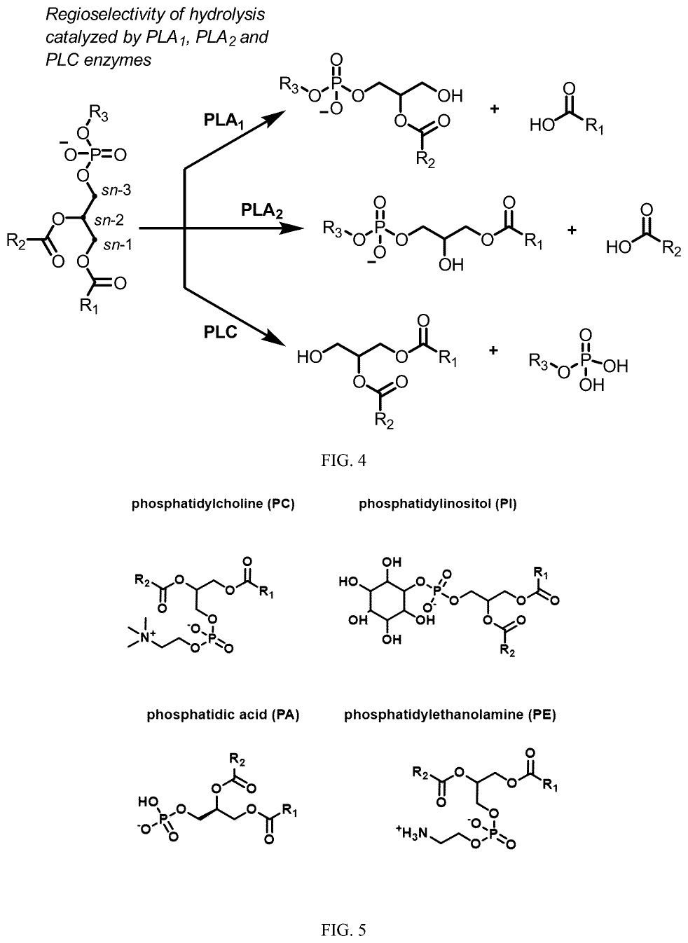

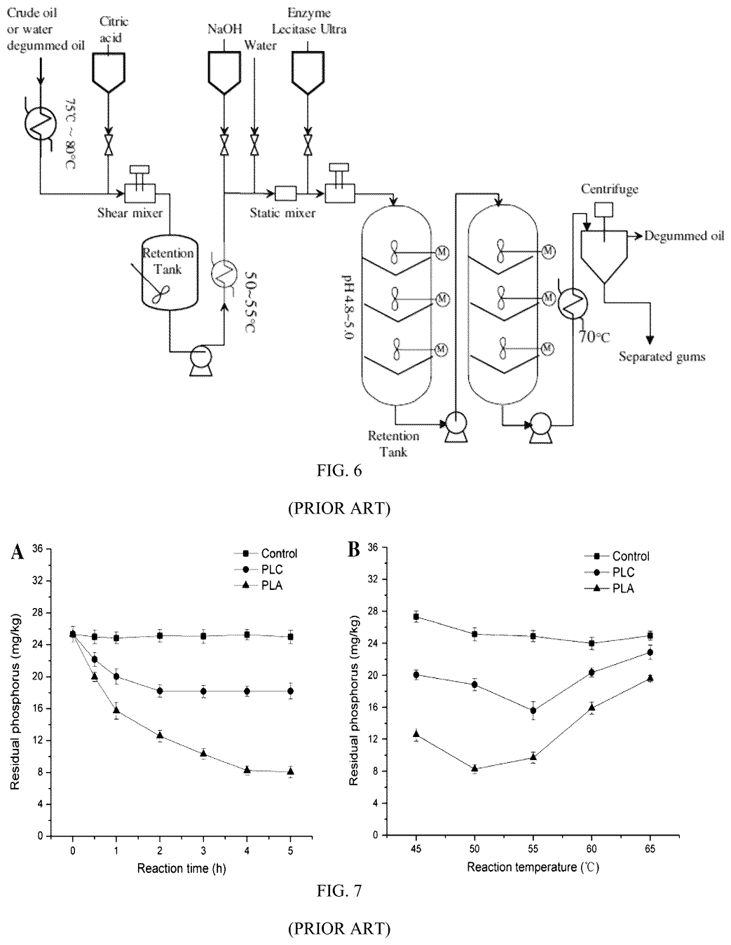

### DETAILED DESCRIPTION OF THE DISCLOSURE

The following is a description of a non-limiting method of the present disclosure used to create exemplary aerogels. Although the claimed subject matter will be described in terms of certain embodiments and examples, other embodiments and examples, including embodiments and examples that do not provide all of the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include all values to the magnitude of the smallest value (either lower limit or upper limit value) and ranges between the values of the stated range.

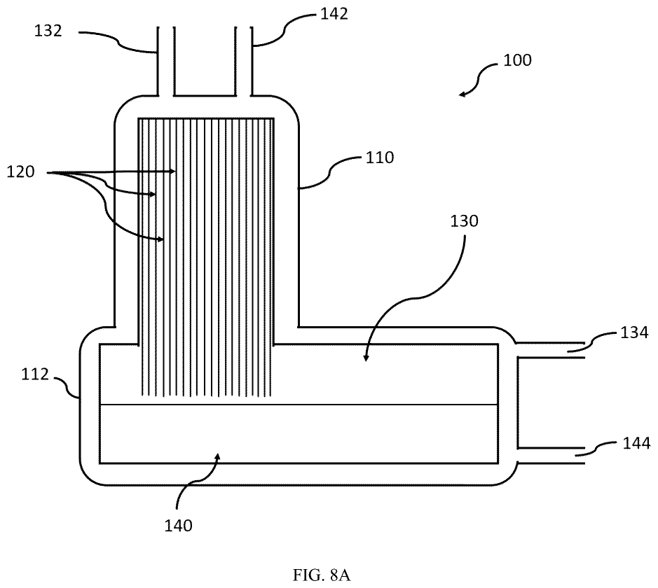

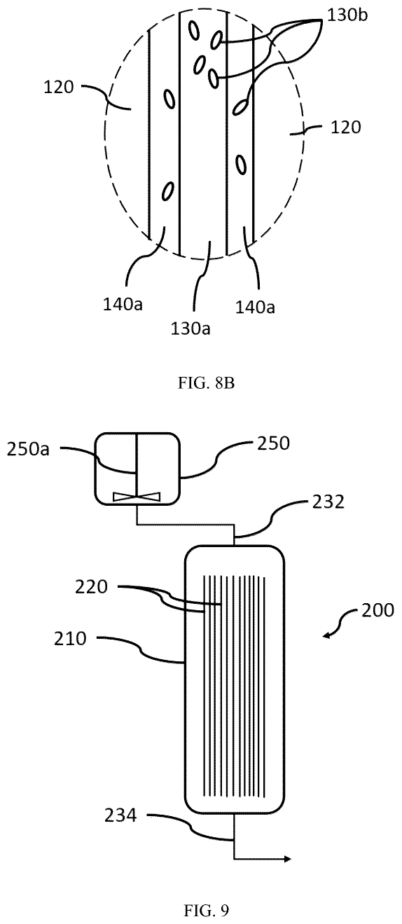

With reference to FIG. 8A, the present disclosure provides a fiber reactor 100 (hereinafter an “immobilized enzyme fiber reactor” or “IEFR”) having internal elements 120 thereof (e.g., fibers) containing immobilized enzymes, namely phospholipases. The IEFR 100 includes a hollow conduit 110 housing a plurality of fibers 120. In some embodiments, the fibers 120 may be formed of steel, such as stainless steel or a steel composite. In other embodiments, the fibers 120 may be formed of basalt, ceramics, glass, or metals. The fibers 120 may extend an entire length of the conduit 110 or a portion thereof, or may extend beyond the conduit 110 as shown in FIG. 8A. In some embodiments, the fibers 120 may be packed into the conduit 110 at a nominal rate of less than 30%, less than 25%, less than 20%, 5 to 30%, about 15%, or about 25%.

The conduit 110 includes one or more inlets 132, 142 for introduction of reactants into the conduit 110. In some embodiments, only a single inlet is present while other embodiments may include 2, 3 or more inlets. The IEFR 100 includes at least one outlet 134, 144 downstream of the inlet(s) 132, 142. In some embodiments, only a single outlet is present while other embodiments may include 2 or more outlets. As shown in FIG. 8A, the IEFR 100 may include a separator tank or settling tank 112. In such embodiments, the separator tank 112 may include an outlet 144 for removing a denser phase 140 (e.g., an aqueous phase) therefrom and an outlet 134 for removing a lighter phase 130 (e.g., an organic phase) therefrom.

In some embodiments, the IERF does not include a settling tank. For example, as shown in FIG. 9, an IEFR 200 includes a conduit 210 with a plurality of fibers 220 disposed therein. The IEFR includes a single inlet 232 in communication with a tank 250, which supplies a mixture to the IEFR 200. The tank 250 may include a mixer 250a. The IEFR 200 also has a single outlet 234 for directing a reacted mixture from the IEFR 200. After exiting the IEFR 200, the reacted mixture may be further processed or directed to a separation process, such as a centrifuge or a drying process. Other configurations of the IEFR may be used, such as those described in U.S. Pat. No. 9,468,866 B2 by Massingill, the entirety of which is hereby incorporated by reference.

The IEFR 100, 200 enables microchannel formation along the fibers 120 to achieve high surface area interfacial bi-phasic diffusion as depicted in FIG. 8A. An oil phase 130a and an aqueous phase 140a cascade down the fiber internals 120 where intimate contact is made between the immiscible phases and the fiber media. Impurities or other targeted components 130b in the oil phase 130a are transferred to the aqueous phase 140a across an interface therebetween. In one or more embodiments, the IEFR 100, 200 provides benefits including high surface area for rapid interfacial diffusion, minimal emulsion formation, an atmospherically sealed system, a small footprint as compared with traditional systems, no moving parts requiring energy and maintenance, and accommodation of a wide range of flow rates.

The present disclosure also provides methods of immobilizing enzymes onto the internal elements of the fiber reactor as well as methods of using the IEFR to enable rapid oil processing for the reduction of phospholipids and metal contents below 1 ppm. The immobilized phospholipases allow the IEFR to achieve continuous impurity removal. Moreover, the enzymes are not consumed during processing through the IEFR allowing one unit of initial enzyme to process many units of oil.

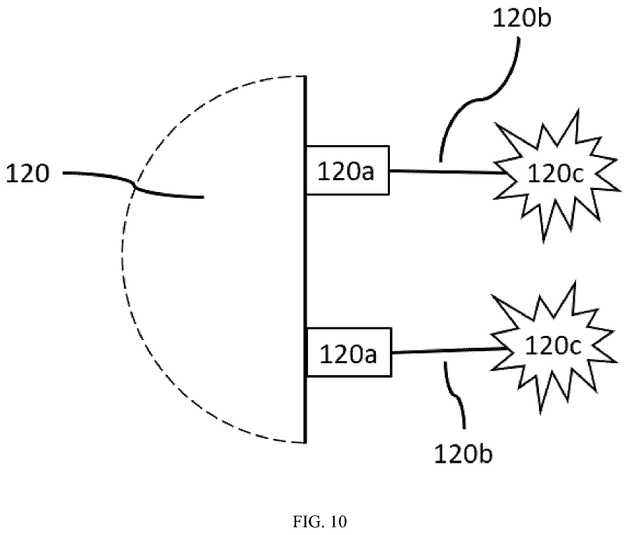

FIG. 10 depicts a fiber 120 having an enzyme 120c immobilized thereon. According to one or more embodiments, the linkage between the fiber 120 and the enzyme 120c may include a plurality of anchor groups 120a bound to the surface of the fiber 120 and a bifunctional crosslinker 120b binding the anchor groups 120a to the enzyme 120c. In some embodiments, the enzyme 120c may be covalently bound to the fiber 120. In such embodiments, a process for immobilizing enzymes on internals 120 of the IEFR 100 (e.g., steel fibers) may include three steps. In the first step, the anchor groups 120a are bound to surfaces of the fibers 120. In some embodiments, the anchor groups 120a comprise an amino compound. For example, an amino silane such as aminopropyl triethoxy silane may recirculated for, e.g., 5-10 minutes against a steel fiber bundle in an ethanolic/water solution. The ethoxy silane groups undergo a hydrolysis-condensation reaction and bind to hydroxyl groups native to the surface. The same would apply to any surface with native hydroxyls (e.g., ceramic, metal, and/or glass). Once bound, the amino groups extend away from the fiber. In another example, a dopamine hydrochloride solution may be recirculated for up to 3 hours or up to 24 hours against the fibers 120 to coat the fibers 120 with polydopamine (as the anchor group 120a). The dopamine hydrochloride solution may include about 0.001 to 10 g/L or about 2 mg/L of dopamine hydrochloride and may be a buffered solution, for example using tris-HCl buffer (pH 7.4) or phosphate buffer (pH 8.5). In any embodiment, the first step may further include rinsing the fibers 120 (e.g., with water) to remove any unbound anchor groups 120a and drying the fibers 120 having the anchor groups 120a attached thereto. In some embodiments, drying may be conducted at elevated temperature (e.g., about 40° C.) and/or under vacuum.

In the second step, an aqueous solution of a bifunctional crosslinker (e.g., glutaraldehyde) is recirculated for, e.g., about 20 minutes against the fiber bundle where the aldehyde groups crosslink with the primary amine of the amino propyl silane. Finally, an aqueous solution of enzyme is recirculated against the fiber bundle for, e.g., about 30 minutes causing the second aldehyde group on glutaraldehyde to react and covalently bond with the amine groups on the enzyme proteins. All of the chemical reagents described above are inexpensive and produced at large, industrial quantities.

Other methods of immobilizing the enzymes onto the fibers may be used, as appreciated by those skilled in the art. For example, the ThermoFisher Scientific Bioconjugation technical handbook Reagents for crosslinking, immobilization, modification, biotinylation, and fluorescent labeling of proteins and peptides provides an extensive list of available linker molecules for the linkage of different functional groups. Table 1 below summarizes some of the linkers useful in the present system, method, and apparatus. In particular, Table 1 lists 21 chemicals for amine-to-amine linkages; 7 chemicals for sulfhydryl-to-sulfhydryl linkages; 28 chemicals for amino-to-sulfhydryl linkages; 3 chemicals for carboxyl-to-amine; 6 chemicals for sulfhydryl-to-carboxyl; 11 chemicals for photoreactive linkages; and 12 chemicals for chemoselective linkages.

TABLE 1
Bioconjugation chemicals available from ThermoFisher Scientific.

| | | |
|---|---|---|
| | | Number of |
| | | different |
| | | chemical |
| | | linkers |
| Functional groups linked | Chemical linker example | available |

Homobifunctional crosslinker

| | | |
|---|---|---|
| Amine-to-amine reactive (NHS ester | DSG (disuccinimidyl glutarate) | 11 |
| Amine-to-amine reactive (NHS | BS2 G-d0 (bis(sulfosuccinimidyl) glutarate-d0) | 6 |
| ester), deuterated or MS-cleavable | | |
| Amine-to-amine reactive | DMP (dimethyl pimelimidate) | 4 |
| (imidoester or difluoro) | | |
| Sulfhydryl-to-sulfhydryl reactive | BMOE (bismaleimidoethane) | 7 |
| (maleimide) | | |

Heterobifunctional crosslinkers

| | | |
|---|---|---|
| Amine-to-sulfhydryl reactive | SIA (succinimidyl iodoacetate) | 4 |
| (NHS-haloacetyl) | | |
| Amine-to-sulfhydryl reactive | AMAS (N-α-maleimidoacetoxysuccinimide ester) | 22 |
| (NHS-maleimide) | | |
| Amine-to-sulfhydryl reactive | SPDP (succinimidyl 3-(2-pyridyldithio) propionate) | 6 |
| (NHS-pyridyldithiol) | | |
| Carboxyl-to-amine reactive | EDC (1-ethyl-3-(3-dimethylaminopropyl)carbodiimide | 3 |
| (carbodiimide plus NHS ester) | hydrochloride | |
| Sulfhydryl-to-carbohydrate or | BMPH (N-β-maleimidopropionic acid hydrazide | 6 |
| -carboxyl (maleimide, | | |
| pyridyldithiol/hydrazide, or | | |
| isocyanate | | |
| Photoreactive (NHS ester and aryl | Sulfo-SANPAH (sulfosuccinimidyl 6-(4′-azido-2′- | 9 |
| azide, phenyl azide, diazirine, or | nitrophenylamino) hexanoate) | |
| psoralen) | | |
| Chemoselective ligation (NHS ester | NHS-Azide | 6 |
| and azide-phosphine or -alkyne) | | |
| Chemoselective ligation | GlcNAz (N-azidoacetylglucosamine, tetraacylated) | 6 |
| Photoreactive amino acids | L-Photo-Leucine | 2 |

Biotin and desthiobiotin labeling reagents

| | | |
|---|---|---|
| Amine-reactive | EZ-Link NHS-Biotin | 12 |
| Sulfhydryl-reactive | EZ-Link BMCC-Biotin | 4 |

Modification reagents for reduction and denaturation of proteins

| | | |
|---|---|---|
| Disulfide bond reduction | 2-Mercaptoethanol/(β-mercaptoethanol) | 6 |
| Schiff base reduction to alkylamine | AminoLink Reductant (sodium cyanoborohydride) | 1 |
| linkage | | |
| Protein denaturants and chaotropes | Guanidine-HCl | 2 |

Modification reagents for proteins and peptides

| | | |
|---|---|---|
| Irreversibly blocks primary amines | Pierce Sulfo-NHS-Acetate (sulfo-N- | 1 |
| | hydroxysulfosuccinimide acetate | |
| Modifies primary amines to contain | Pierce SATP (N-succinimidyl S-acetylthio-propionate) | 3 |
| a protected sulfhydryl group | | |
| Modifies primary amines to contain | Traut's Reagent (2-iminothiolane) | 1 |
| a free sulfhydryl group | | |
| Adds amine or carboxylic acid | Pierce AEDP (3-((2-aminoethyl)dithio) propionic acid- | 1 |
| functional group to protein or | HCl) | |
| surface | | |
| Irreversibly blocks sulfhydryl | Pierce NEM (N-ethylmaleimide) | 1 |
| groups | | |
| Reversibly blocks sulfhydryl groups | Pierce MMTS (methyl methanethiosulfonate) | 1 |
| Adds primary amine to glass and | Pierce APTS (3-aminopropyltriethoxysilane | 1 |
| silica surfaces through silylation | | |
| Oxidizes carbohydrates for | Pierce Sodium Meta-Periodate | 1 |
| reductive amination | | |
| Alkylates reduced cysteines | Pierce Iodoacetic Acid | 3 |
| Deprotects SATA-modified | Pierce Hydroxylamine-HCl | 1 |
| molecules | | |

PEGylation (PEG labeling) reagents for proteins

| | | |
|---|---|---|
| Amine-reactive linear PEGylation | MS(PEG)4 (methyl-PEG4-NHS ester) | 4 |
| of protein or surface, terminating | | |
| with a methyl group | | |
| Amine-reactive branched | TMS(PEG)12 ((methyl-PEG12) 3-PEG4-NHS ester | 1 |
| PEGylation of a protein or surface, | | |
| terminating with a methyl group | | |
| Sulfhydryl-reactive branched | MM(PEG)12 (methyl-PEG12-maleimide) | 2 |
| PEGylation of a protein or surface, | | |
| terminating with a methyl group | | |
| PEGylation of a protein or surface, | CA(PEG)4 (carboxyl-(4-ethyleneglycol) ethylamine | 4 |
| terminating with a carboxylic acid | | |
| or primary amine | | |
| PEGylation of a gold, silver, or | CL(PEG)12 Carboxy-PEG-Lipoamide Compound | 3 |
| metal surface, terminating with a | | |
| carboxylic acid or methyl group | | |
| PEGylation of a protein or inert | MT(PEG)4 Methyl-PEG-Thiol Compound | 1 |
| material surface, terminating with | | |
| a methyl group | | |
| PEGylation of a protein, oxidized | CL(PEG)12 Carboxy-PEG-Lipoamide Compound | 3 |
| carbohydrate, or surface, | | |
| terminating with a methyl group | | |
| PEGylation of a protein or inert | MT(PEG)4 Methyl-PEG-Thiol Compound | 1 |
| material surface, terminating with | | |
| a methyl group | | |
| PEGylation of a protein, oxidized | MA(PEG)4 (methyl-(4-ethyleneglycol) ethylamine) | 4 |
| carbohydrate, or surface, | | |
| terminating with a methyl group | | |

Fluorescent dye labeling reagents and kits

| | | |
|---|---|---|
| Amine-reactive | Alexa Fluor 350 NHS Ester (Succinimidyl Ester) | 65 |
| Sulfhydryl-reactive | Alexa Fluor 350 C5 Maleimide | 18 |
| Carboxyl-reactive | Alexa Fluor 350 Hydrazide | 20 |
| Chemoselective | Alexa Fluor 488 Azide | 28 |

In some embodiments, the enzyme 120c may be bound without using the bifunctional crosslinker 120b. For example, the enzyme 120c may be bound to the fiber 120 using Van der Waals forces. In such embodiments, the fibers 120 may first be cleaned of any oils or organic compounds by circulating a suitable organic solvent such as hexane or denatured alcohol or ethanol against the fibers 120 for a period of time, for example, about 20 minutes. After the fibers 120 are cleaned the solvent may be rinsed to remove non-polar organic solvent. A 95/5 (by volume) ethanol (or denatured alcohol) and water solution is acidified to a pH of about 4.5 with an acid such as acetic acid. The acid may act to accelerate hydrolysis-condensation reactions. To this mixture, about 1 to 5 vol % of trimethoxyhexadecyl (or octadecyl or octyl) silane is added, and the resultant mixture is recirculated for a period of time (e.g., about 10 minutes) over the fibers 120. The fluid is drained and the fibers 120 are rinsed with denatured alcohol to remove loosely bound alkyl silane. The fibers may then be dried (e.g., at about 110° C. for about 10 minutes) to drive silane crosslinking to completion. The enzyme 120c may then be circulated over the alkyl functionalized fibers for about 20-30 minutes to bind the enzyme 120c to the fibers 120. The fibers 120 may be rinsed with DI water to remove any unbound enzyme. In some embodiments, the enzyme 120c may be PLA or PLC and circulating the enzyme 120c over the fibers 120 may be accomplished using a mixture of 1 ml of enzyme 120c per 1 L of water.

Once the enzymes are bound to the column, they are active for treating oil containing phospholipids, such as DCO or SBO. In some embodiments, the oil may be pretreated with aqueous citric acid (approximately 3% added water) for, e.g., about 15 minutes in a stirred beaker at, e.g., about 80° C. In some embodiments, the pH may be adjusted between 4.1-5.1 (i.e., an ideal activity pH range of the specific enzyme used as example) with aqeuous sodium hydroxide (approximately 0.1% added water) and the temperature of the oil may be reduced to between 50-55° C. (i.e., an ideal activity temperature range of the specific enzyme uses as example). In other embodiments, the pH and temperature ranges may be tailored to the enzymes used. The oil may then be introduced into the IEFR and contacted against a second water stream entering the IEFR.

Because the action of enzymatic degumming occurs at the oil-water interface, the unique mixing function of the IEFR, where micro thin films are formed along the fiber internals, allows for intimate and high-surface area contact between the oil and aqueous phases. Due to this effect, phospholipids and associated metals can be reduced below 1 ppm in a matter of a few minutes after cascading down approximately 4 feet of IEFR internals. Additionally, the enzymes maintain their functionality and are not washed away from the fiber surface allowing continuous processing without having to add additional enzyme with each fresh batch of crude SBO. A second effect is that the FFAs liberated from the phospholipids add to the amount of lipid mass residing in the oil. This is an added benefit for renewable fuels processing as the cleaved phosphates will be washed away with the water while the cleaved FFAs will remain with the oil and add to the total feedstock yield unlike in water-acid degumming alone thereby increasing total yield potential for fuel production.

For traditional immobilized enzymatic solutions using packed bed media, once the enzyme activity has decreased, the packed bed is discarded, and new packing is purchased with pre-immobilized enzyme. This process is costly and involves downtime during which the process cannot be conducted. In contrast, the presently disclosed IEFR is capable of reuse without discarding the fibers. In particular, if the enzyme activity has decreased or if there is a desire to change to type of enzymes on the IEFR, the fibers can be stripped of the enzyme and new enzymes can be applied according to the methods disclosed herein. In some embodiments, the fibers can be cleaned of residual oils with organic solvent then stripped of the immobilized enzymes by circulating a strong base (e.g., NaOH) and an alcohol through the IEFR. Although according to some embodiments the enzymes can be immobilized onto the fibers before installing the fibers in the IEFR, it is also possible to remove and apply enzymes from the fibers without deconstructing the IEFR. This provides substantial advantages in terms of reduced costs and reduced downtime as compared with traditional systems.

The present disclosure further relates to a method of treating an oil containing phospholipids using the IEFR disclosed herein. The treatment method may include the immobilization method described above as an initial step to prepare the IEFR with phospholipase on the fibers thereof. The treatment method may also include an oil pretreatment, as described above. For example, the pretreatment step may include stirring the oil for about 15 minutes at 80° C. with an aqueous solution containing a chelating acid group (e.g., citric acid or phosphoric acid). In some embodiments, the pretreatment may be conducted at room temperature or at an elevated temperature of about 40-90° C., about 50-90° C., about 60-85° C., about 70° C., or about 80° C. The chelating acids will allow a significant portion of the phosphatidic acids which are coordinated to divalent metals such as magnesium and calcium to be hydrated. Then the pH of the oil is adjusted to pH 4-5 where the enzyme is active. This may be achieved using a base, such as NaOH. Below pH 3, the histidine residues at the active site of the enzyme may become protonated and functionality may be reduced. In the absence of a pretreatment step, the metal impurities will not be removed as well compared to utilizing a pretreatment step.

In some embodiments, the pretreatment adds about 0.1 to 10 wt %, about 0.2 to 8 wt %, or about 0.4 to 3 wt % of water (an aqueous solution of the chelating acid and the buffering base) to the oil. In some embodiments, after pretreatment, the pretreated oil may be separate from the water. Additional water is then added to the separated pretreated oil as described below. In other embodiments, the pretreated oil may be introduced into the IEFR as a mixture of the pretreated oil with the aqeuous phase. In such embodiments, additional water may optionally be added to this mixture. When the pretreatment water is left in the oil, the total water content described herein refers to the amount of water added in the pretreatment step and the additional water added to the oil before or during introduction into the IEFR.

As discussed above, the enzymatic reactions occur at an interface between water, oil, and the enzyme (phospholipase). As such, the treatment method includes introducing water and oil into the IEFR, the IEFR having enzymes immobilized on the fibers thereof. Surprisingly, it was found that low impurity values (in the treated oil) can be attained using as little as 3 wt % water (or even less) based on the total mass of the oil and water supplied to the IEFR. This low water content provides significant advantages including lower pressure drop, higher throughput of oil, and less waste water to be disposed of. As used herein, the water used in the treatment method may consist of water or may include additives such as acids, bases, or salts.

In some embodiments, the water and oil are separately introduced into inlets of the IEFR. In such embodiments, a ratio between a flow rate of the oil and a flow rate of the water may be 85:15 to 99:1, at least 85:15, at least 87:13, at least 90:10, at least 92:8, at least 95:5, or about 97:3. In other embodiments, the water and the oil are pre-mixed and supplied to the IEFR through a single inlet. In such embodiments, a weight ratio of the oil to the water may be 85:15 to 99:1, at least 85:15, at least 87:13, at least 90:10, at least 92:8, at least 95:5, or about 97:3.

After introduction, the oil and water react in the presence of the enzyme and a least a portion of the phospholipids within the oil are digested to form water-soluble phosphate groups that migrate into the water. In other words, contacting the water and the oil with the enzymatic fibers of the IEFR reduces a phosphorous content of the oil. The contacting may also remove other impurities, such as metals including magnesium and calcium, from the oil into the water. Some of these metals may be bound within the phospholipids and released by the aforementioned enzymatic digestion of the same.

In some embodiments, the oil and water may be reacted at room temperature or an elevated temperature of, for example, 30-80° C., 40-70° C., 50-60° C., 50-55° C., about 50° C., about 55° C., or about 60° C. Depending on the enzyme used, the temperature may be appropriately adjusted to an optimal temperature range thereof. In some embodiments, the reaction is conducted at a reduced pH of less than 7, less than 6, about 4 to 6, about 4 to 5, about 4, about 4.5, or about 5. As with the temperature, the pH may be controlled to an optimal range for the enzyme or enzymes used in the IEFR. In some embodiments, the pH is controlled via the pretreatment of the oil, as discussed above. In other embodiments, the pH may be controlled by modifying the water with an acid or a buffered solution of acid and base.

Once the treated oil and effluent (water having impurities from oil dissolved therein; the effluent may further comprise undissolved solids) exit the fibers of the IEFR, they may enter a separator tank and be separately withdrawn therefrom. Due to the nature of the IEFR, the treated oil and effluent readily disengage. In other embodiments, the treated oil and effluent may be further treated or may be directed to an alternative separation process, such as a centrifuge.

In any embodiment, a pressure within the IEFR may be maintained at about 1 to 125 psi, about 1 to 100 psi, about 1 to 50 psi, or about 20 psi. In some embodiments, a total flow rate of the oil and water per cross-sectional area (cm²) of the IEFR (whether pre-mixed or separately supplied) may be at least 10 ml/min/cm², at least 15 ml/min/cm², at least 25 ml/min/cm², at least 50 ml/min/cm², at least 75 ml/min/cm², at least 100 ml/min/cm², at least 125 ml/min/cm², at least 150 m/min/cm², or 20 to 250 ml/min/cm². The IEFR may be readily scaled to accommodate industrial quantities of oil. Moreover, the contact time needed to effectuate the reactions described herein is drastically reduced as compared to traditional methods (see discussion of FIG. 7 above). The IEFR also facilitates quick separation of the treated oil from the effluent, whereas traditional stirred reactions involve long settling times and additional infrastructure to facilitate the same.

Advantages of the IEFR and oil refining processes using the same include removal of phospholipids and metal ions from crude vegetable oil (e.g., SBO) without caustic refining, removal of phospholipids and metal ions from crude vegetable oil without the requirement of centrifugation equipment, recovering free fatty acids normally lost from phospholipids, wherein the free fatty acids cleaved off phospholipids add to the lipids convertible to renewable diesel and SAF thereby increasing energy yield per unit SBO processed, reduction of need for exogenous phospholipase enzymes for enzymatic refining of vegetable oil, reduction of agitation energy and mixing time required for enzymatic refining, and reuse of the column and fiber packing by removing enzymes from the fibers and reapplying enzymes to the same column.

### EXAMPLES

### Example 1: Immobilization of Enzyme onto Fibers

Fibers were functionalized in a plastic tube having a diameter of approximately 1″. The tube was a recirculation tube with a screwed fitting at the bottom to introduce fluids which then flowed to the top and flowed back into the fluid vessel. A stopper was fitted snugly into the top of the tube to seal the top and hold the exit tubing. The stopper also included an eye hook attached to the underside thereof to connect to the the top of a fiber bundle to affix it during operation. Plastic tubes are preferred for functionalization as the silane will bind to glass.

Denatured alcohol or ethanol was recirculate against the bundle to remove any oils/organics that are found on the steel fiber for approximately 20 minutes. This alcohol can be reused multiple times. After several uses, a very slight yellow tinge becomes apparent from the removed oils, at which point the rinse alcohol may be replaced.

A 95 vol % EtOH (or denatured ethanol solution) and 5 vol % H₂O solution was mixed with 2 vol % of aminopropyl triethoxy silane and added to the tube, such that the tube was completely filled (about 500 to 1000 ml). The solution was recirculated for about 5 minutes on the fiber bundle and then the fluid was drained.

The bundle was then rinsed with fresh denatured alcohol to remove loosely bound amine silane. The bundle was dried at about 80° C. for 1 hour in an oven to drive silane crosslinking to completion. The bundle was pulled back into the functionalization tube and an aqueous solution of 0.25% glutaraldehyde was prepared and recirculated over the bundle for 30-45 minutes. The solution was drained and the bundle was rinsed with fresh water to remove unbound glutaraldehyde. An enzyme solution was prepared by mixing (a) 100 ppm of PLA₁ in distilled water, (b) 100 ppm of PLA₁ and 1000 ppm of PLC in distilled water, or (c) 1000 ppm of PLC in distilled water. The enzyme solution was recirculated over the glutaraldehyde fuctionalized silane bundle for about 20-30 minutes. The fiber bundle was then rinsed with deionized water to remove unbound enzyme.

### Example 2

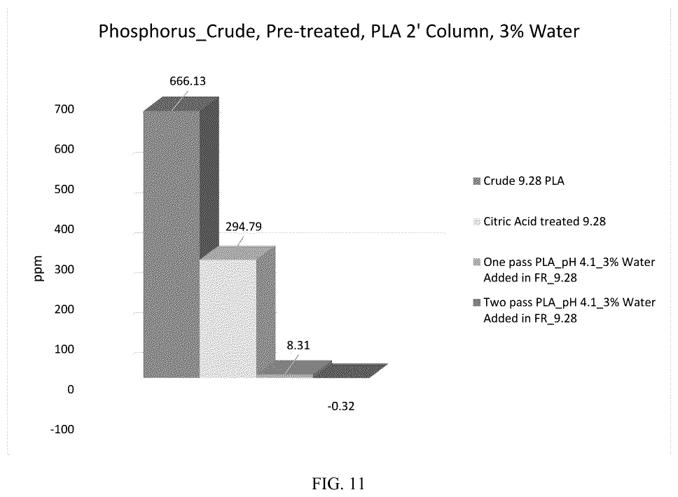

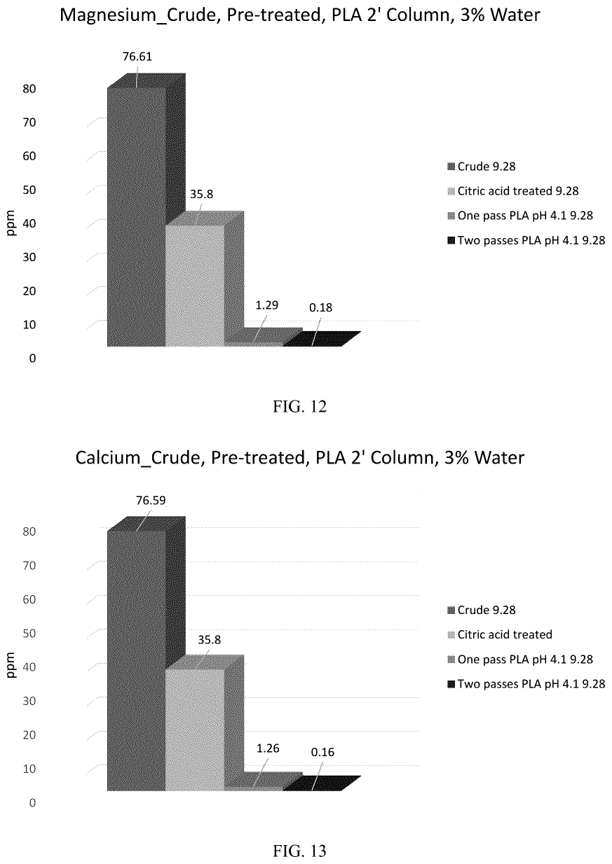

Crude SBO was pretreated with citric acid and then passed through a 2 foot long IEFR having PLA enzyme immobilized on the internals thereof. The phosphorous, magnesium, and calcium contents were measured for the crude SBO, the SBO after citric acid pretreatment, the SBO after pretreatment and a single pass through the IEFR, and the SBO after a second pass through the IEFR (4 feet reaction length). The results are shown in FIGS. 11-13, wherein the phosphorous, magnesium, and calcium contents precipitously fell after the first and second IEFR treatments.

### Example 3

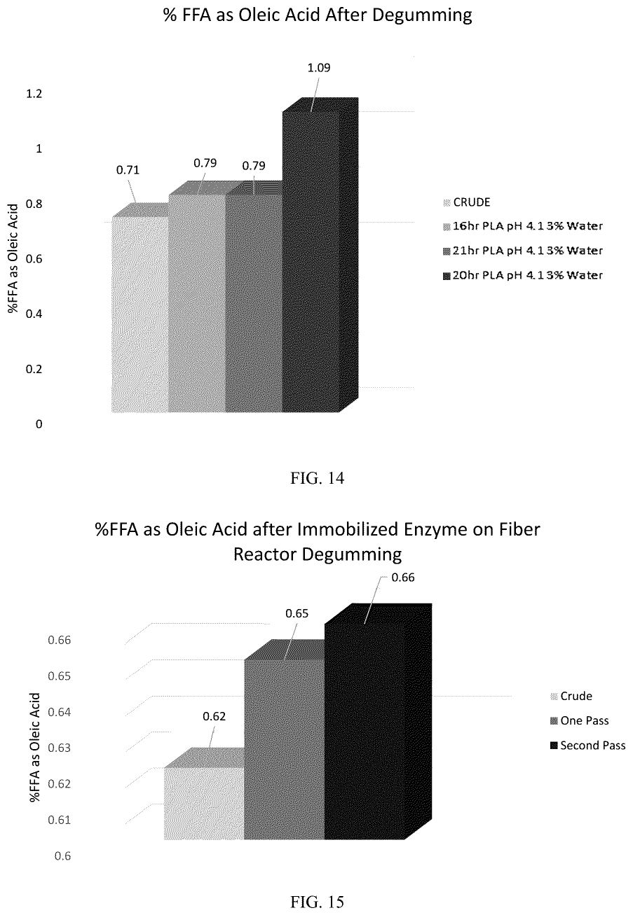

Crude SBO was pretreated with acid to a pH of 4.1 and contacted with 3% water in an IEFR having PLA₁ immobilized thereon. The contact time was varied via recirculation as shown in FIG. 14. As shown in FIG. 14, the treatment with PLA₁ increased the FFA content in the treated oil as compare with the crude SBO.

### Example 4

Crude SBO was pretreated with acid to a pH of 4.1 and contacted with 3% water in an IEFR having PLA₁ immobilized thereon. Two passes were conducted and, as shown in FIG. 15, the treated oil had increased FFA content as compared with the crude SBO.

### Example 5

1000 mL Crude SBO was heated to 80° C., 30% citric acid solution was added at 1 g citric per kg oil, and the mixture was stirred for 15 minutes. A sufficient amount of 14% NaOH solution was added to bring pH to 4.1. The temperature was brought to between 50-55° C. Oil was introduced into a PLA1 modified IEFR (2′×1″ with a bundle formed of 16,500 loops BEKAERT fiber—a nominally 13% pack) and contacted against water that was simultaneously introduced. The water flowed at 3 mL/min (30 mL water) and the oil at 100 mL/min until all the water and oil had run through the column. 3% total water was added.

The speed was reduced to 50 mL/min once all the oil had collected into the collection beaker after one pass. A sample was collected off the bundle after the first pass.

The enzyme treated oil was then introduced to the column again without the addition of additional water for a total of 6 passes. A sample was collected off the bundle after each pass.

The samples were analyzed for phosphorous and total metal content and the results are summarized in Table 2 below.

TABLE 2

| | | Total Metals |
|---|---|---|
| Sample Type | Phosphorus | (P, Mg, Ca, K, Fe) |
| Crude | 666.13 | 1054.41 |
| Acid Pretreated | 294.79 | 478.31 |
| First Pass | 8.31 | 16.71 |
| Second Pass | <DL | 4.11 |
| Third Pass | <DL | 4.10 |
| Fourth Pass | <DL | 4.17 |
| Fifth Pass | 0.49 | 4.37 |
| Sixth Pass | 0.22 | 4.51 |

After the second pass, most notable impurities fall below or at the detection limit of the instrument (“<DL” in Table 2 denotes values below the detection limit). The element that remains after a second pass and makes up the vast majority of the total metals count is potassium (K).

### Example 6

Crude SBO was pretreated with citric acid (1 g citric per kilogram oil treated stirred for 15 minutes at 80° C.). The pH was adjusted to between 4.2 and 4.5 with concentrated sodium hydroxide and then the pretreated oil was passed through an IEFR with reverse osmosis (R.O.) water (3% water added). The IEFR was 5′ long with a 0.16″ diameter filled with crimped fiber (one 5′ length of crimped tow, 550 strands; nominally 8.6% pack) and the oil was introduced into the IEFR at a speed of 21 mL/min. The crude SBO had an average phosphorous content of 586 ppm and an average total metal content of 962 ppm. The pretreated SBO had an average phosphorous content of 50 ppm and an average total metal content of 96 ppm.

Additional SBO samples were not pretreated but were instead passed through the IEFR with a citrate buffer solution (citric acid+sodium citrate; pH adjusted between 4.2 and 4.5). A range of citrate concentrations were explored with the citrate buffer solution from 0.63 to 3.15 g citrate per kilogram oil. However, as summarized in Table 3 below, increasing the concentration well above that of the standard citric acid pretreatment at 1 gram citrate per kilogram oil did not produce an oil comparable to that utilizing a chelating acid pretreatment followed by the enzymatic fiber reactor.

TABLE 3

| | Pretreatment, R.O | Pretreatment, R.O. | No pretreatment, | No pretreatment, |
|---|---|---|---|---|
| Mass | water aqueous, | water aqueous, | citrate buffer | citrate buffer |
| citrate (g) | Phosphorus | Total Metals | aqueous, | aqueous, Total |
| per oil (kg) | (ppm) | (ppm) | Phosphorus (ppm) | Metals (ppm) |
| 0.63 | | | 61.0 | 123.1 |
| 1 | 6.3 (avg value) | 11.4 (avg value) | | |
| 1.58 | | | 34.4 | 73.7 |
| 2.36 | | | 43.9 | 88.5 |
| 3.15 | | | 40.2 | 81.7 |

### Example 7

Pretreatment with chelating acids significantly reduces the concentration of non-hydratable phospholipids allowing more efficient enzyme treatment to reduce the final phosphorus value below 10 ppm. Similar pretreatment procedures as those outline in Example 6 above we repeated. An additional sample was pretreated with citric acid (as above) and 3% water vortexed for 10 second. Table 4 below summarizes the phosphorous and total metal contents before and after pretreatment.

TABLE 4

| | Phosphorus | Total Metals |
|---|---|---|
| Oil Type | (ppm) | (ppm) |
| Crude Soybean Oil | 216.50 | 364.53 |
| Pretreated with Citric Acid | 59.45 | 106.70 |
| Pretreated with Citric Acid | 19.37 | 29.13 |
| and 3% Water Vortex | | |

### Example 8

Crude SBO was pretreated as detailed in Example 6 above and the pH was adjusted to between 4.5 and 5. Each sample was passed through an IEFR under varying conditions as detailed below below. Each IEFR was 2′ in length, had a 0.6″ ID, and PLA₁ immobilized on the fibers, and the oil was passed at 50° C. in all cases.

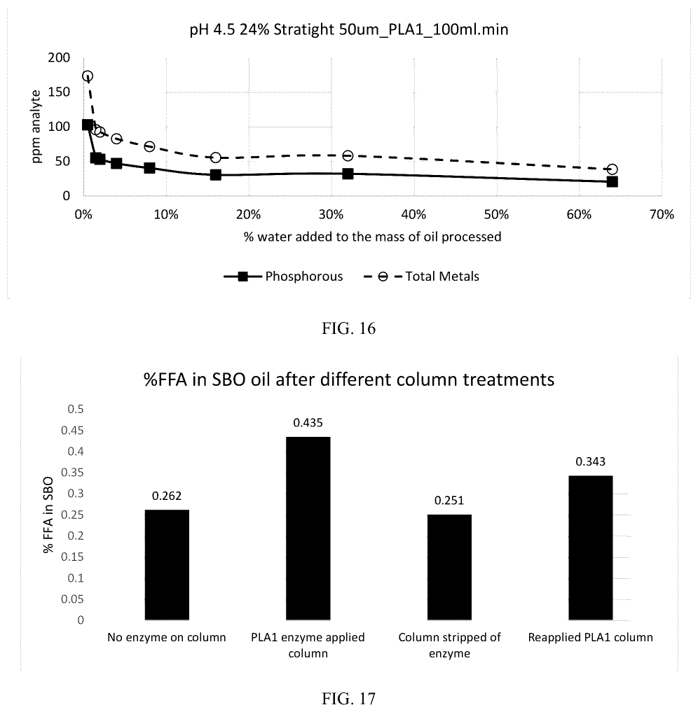

First, samples were passed through an IEFR having a nominally 24% pack and straight 50-micron enzymatic fibers. As shown in Table 5 below, phosphorous and total metals remained high at 100 mL/min oil flow with 3% relative water added through the fiber column. Between 0.5% water added and 64% water to oil (64 g water to 100 g oil) added, the phosphorus value dropped from 102.93 ppm to 20.41 ppm. The results are also shown in FIG. 16.

TABLE 5
pH 4.5 24% Straight 50 μm_PLA_{1 —}100 ml · min

ppm concentration

| Water amount | Phosphorous | Total Metals |
|---|---|---|
| 0.50% | 102.93 | 173.55 |
| 1.50% | 55.04 | 95.87 |
| 2% | 52.91 | 92.39 |
| 4% | 46.98 | 82.64 |
| 8% | 40.27 | 71.41 |
| 16% | 30.47 | 55.37 |
| 32% | 31.99 | 58.09 |
| 64% | 20.41 | 38.41 |

As shown in Table 6 below, when the same column is operated at 300 mL/min, the phosphorus drops to 5.51 ppm at 3% water, 1.68 ppm at 50% water to oil ratio, and 1.45 ppm at 100% water to oil ratio (100 g water to 100 g oil).

TABLE 6
pH 4.5 24% Straight 50 μm PLA₁ 300 mL/min

ppm concentration

| Water amount | Phosphorous | Total Metals |
|---|---|---|
| 3% | <DL | <DL |
| 50% | <DL | <DL |
| 100% | <DL | <DL |

Next, samples were passed through an IEFR having a nominally 15% pack and crimped 50-micron enzymatic fibers. As summarized in Table 7 below, the phosphorus value attained were below the detection limit of the inductively coupled plasma (ICP) (<DL) at 3% water and 300 mL/min oil flow and were found to be below detection using 25-100% water as well. This indicates that the additional water consumption is not warranted.

TABLE 7
pH 5 15% Crimped 50 μm PLA₁ 300 ml/min

ppm concentration

| Water amount | Phosphorous | Total Metals |
|---|---|---|
| 3% | <DL | <DL |
| 25% | <DL | <DL |
| 50% | <DL | <DL |
| 100% | <DL | <DL |

From the foregoing, it appears at the lower oil flow rate in a less packed column (24% straight 50 micron fiber), there was not sufficient mixing with the water even as relative water used increases substantially. However, when the oil rate was tripled, the impurity values dropped significantly even at 3% water usage and gradually declined as substantial amounts of water is added. As such, the speed of oil flow appears to be a much more significant factor than the need for copious amounts of water.

### Example 9

Crude SBO was pretreated as detailed in Example 6 above and the pH was adjusted to between 4.5 and 5. This oil flowed through an IEFR at 50° C. while contacting 3% relative to oil R.O. water. All trials were conducted on a 2′ by 0.6″ ID IEFR with either a nominal 24% straight 50-micron fiber, a nominal 15% straight 50-micron fiber, or a nominal 15% crimped 50-micron fiber. Unless otherwise noted, the fiber was immobilized with PLA₁. The oil speed flow rates and accompanying water flow rates were varied.

As shown in Table 8 below, values for the phosphorous could be brought to 8.49 ppm for the 24% straight 50-micron packing but only after flowing at 300 mL/min oil.

TABLE 8
pH 4.5 24% Straight 50 μM PLA₁ 3% water

ppm concentration

| mL/min flow rate | Phosphorous | Total Metals |
|---|---|---|
| 50 | 28.11 | 42.13 |
| 100 | 26.64 | 39.05 |
| 200 | 9.85 | 13.64 |
| 300 | <DL | <DL |

As shown in Tables 8 and 9, at 100 mL/min oil flow, the phosphorus values for the 15% and 24% straight 50-micron fiber where 28.09 ppm and 26.64 ppm, respectively. By contrast, the 15% crimped 50-micron fiber returned a phosphorus value below the detection limit of the ICP when operated at 300 mL/min oil flow while a similarly packed column with no enzyme gives a final phosphorus value of 9.7 ppm.

TABLE 9
PLA₁ enzyme compared to NO ENZYME 3% water

| | ppm concentration | |
|---|---|---|
| 15% pack, 50 μm fiber | Phosphorous | Total Metals |
| straight, 100 mL/min, PLA₁ enzyme | 28.09 | 58.49 |
| crimped, 300 mL/min, PLA₁ enzyme | <DL | <DL |
| crimped, 300 mL/min, NO ENZYME | 9.7 | 13.9 |

### Example 10

Phospholipase with PLC enzymatic hydrolysis pattern was immobilized onto a fiber reactor with method described in Example 1. Crude SBO containing impurities listed in Table 10 below was treated to increase dialcylglycerol (DAG) content of the crude soybean oil. In particular, the crude SBO was passed concurrently against a sodium phosphate buffer at pH7 through a static mixer and then entering the immobilized PLC on an IEFR that was held at 50° C. The aqueous buffer was added at 3% by volume relative to the oil. The DAG content of the crude oil mixed with the pH 7 buffer before and after one pass through the IEFR was analyzed by GCMS to determine the increase in DAG content. The DAG content measured in the crude oil mixed with pH 7 buffer was 0.310% and after going through the enzymatic column had risen to 0.695% which is an increase of 0.385%. To accurately predict the theoretical yield for DAG generation if all phospholipids were hydrolyzed by PLC action the total phosphorus content as measured by ICP (which will include phosphorus from phospholipids and other phosphorus containing species) and phosphorous NMR (PNMR) to determine the total amount of each phospholipid type must be used. The PLC enzyme will primarily act on the phosphotidyl choline and phosphotidyl ethnolamine types as they are the most abundent, so knowing the exact content of these two species will give the most accurate estimation of DAG theoretical yield. The ICP results will include phosphorus arising both from phospholipids and other phosphorus containing species. The non-phospholipid phosporous containing moieties will not contribute to the yield. At the present, only ICP results are available for the crude soy bean oil used in this example, so a rough approximation can be made from this value using the following formulas.

$$PL ( \% ) = \frac{25 \times P ( \frac{m g}{k g} )}{10 , 000}$$

$$DAG \% = ( P C + P E + Pl ) \times \frac{617}{779} \times 0.85$$

Using the 513 mg/kg phosphorus content measured by ICP for the crude SBO and shown in Table 10 below, the PL % (phospholipid content) is calculated to be 1.28%. Substituting this percentage into the DAG calculation to substitute for PC, PE and PI, the theoretical DAG yield is calculated to be 0.86%. However, due to the reasons expressed above, this DAG % is inflated as it is assuming all phosphorus content measured by ICP is arising from phospholipids. Based on this theoretical yield the actual yield measured by GCMS was found to be 0.385% which would be 48% of the expected yield from one pass through the enzymatic reactor.

TABLE 10

| | Crude Soybean Oil |
|---|---|
| Metal Contaminant (ppm) | (not centrifuged) |
| Phosphorous (P) | 513 |
| Magnesium (Mg) | 40 |
| Calcium (Ca) | 32 |

The PLC enzyme only hydrolyzes phosphatidyl choline (PC) and phosphatidyl ethanolamine (PE). Therefore, any other phospholipids present (e.g, phosphatidyl inositol (PI), phosphatidyl serine (PS) or phosphatidic acid (PA)) will remain intact and solubilized within the oil. This becomes obvious in noting that the metal impurities typically associated with phospholipids (K, Mg, Ca) which remain in the oil after enzymatic treatment if added together total 13.72 ppm which is 1:1 with the remaining phosphorus impurity which totals 13.24 ppm indicating the remaining phospholipids are those associated with metals. Distributions of these phospholipids are known to those skilled in the art. The hydrolysis of PC and PE typically requires 2 hours in a stirred PLC enzymatic procedure (see, e.g., discussion of FIG. 7). The oil impurities that remain after one pass through the PLC immobilized fiber reactor column are obtained with a transit time of ˜10-30 seconds through the column.

### Example 11

An IEFR with PLA₁ immobilized thereon was compared with a non-enzymatic fiber reactor. The reactor was 2′ long with an ID of 0.62″ having a 15% pack of crimped fiber. Oil was flowed at 300 mL/min. The experiments were conducted between 40-50° C. and pH 4-5. The SBO was pretreated by stirring for 15 minutes at 80° C. with a citric acid with the addition of 3% water to oil volume followed by pH adjustment to between pH 4-5 with NaOH.

The pretreated oil was passed through a reactor with no immobilized enzyme. The reactor was then modified by immobilizing PLA₁ thereon as described in Example 1 (to make an IEFR) and another sample of pretreated oil was passed through the IEFR. Next, the PLA₁ enzyme coating was removed by washing the fiber bundle with 4% NaOH in ethanol/water solution containing 8% water for one hour at room temperature. Another sample of pretreated oil was passed through the newly stripped column with no immobilized enzyme. Finally, the column was rinsed with ethanol to remove oil and the enzyme coating was reapplied for the last experiment where pretreated oil was passed over the same column with the reapplied PLA₁ enzyme. All samples were centrifuged, and the top layer of oil analyzed for free fatty acid content.

As can be seen in FIG. 17, the FFA content rises during processing of the SBO through both the firstly applied PLA₁ immobilized enzymatic reactor and the reapplied PLA₁ immobilized reactor compared to the two columns that do not have enzyme labeling. This result demonstrates that the enzyme can be removed and reapplied in a field application without replacing the column packing. This is a substantial advantage over traditional immobilized enzymatic solutions using packed bed media as once the enzyme activity has decreased, the packed bed is discarded, and new packing is purchased with pre-immobilized enzyme. The cost of throwing away the packing with the enzyme is more expensive than just reapplying the enzyme onto the same enzyme support structure.

In FIG. 17, the PLA₁ immobilized and reapplied PLA₁ immobilized columns yield a positive increase in % FFA between the null and the enzymatic experiments. In contrast, no increase in % FFA is seen with the stripped column which had the enzyme coating removed.

### Example 12

FFA content of treated SBO was measured as a function of reactor type (IEFR v non-enzymatic reactor) and temperature. Each reactor used was 2′ long with an ID of 0.62″ having a 15% pack of crimped fiber. Oil was flowed at 300 mL/min. All SBO samples were pretreated the same with citric acid and 3% water addition for 15 minutes at 80° C. After pretreatment, the pH was adjusted to 4.5. All samples were centrifuged, and the top layer of oil analyzed for free fatty acid content. The IEFR with PLA₁ was prepared as described in Example 1.

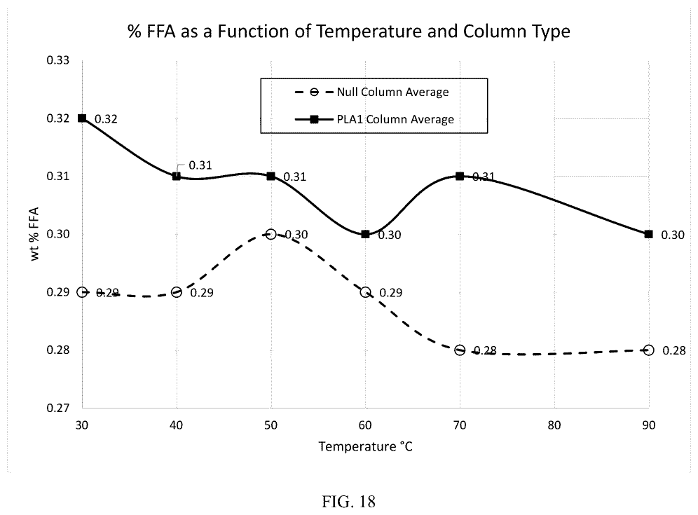

The pretreated oil was brought to the specified temperature and then immediately flowed through the column with one pass and this sample was collected for analysis. Two experiments were conducted for both the non-enzymatic reactor (same column both times) and the IEFR (two different columns). The % FFA value was averaged between these two experiments and is shown in FIG. 18. As shown in FIG. 18, the IEFR provided increased % FFA over the non-enzymatic reactor at all temperatures within the range studied.

Although several embodiments have been disclosed in detail above, the embodiments disclosed are not limiting, and those skilled in the art will readily appreciate that many other modifications, changes, and substitutions are possible in the disclosed embodiments without materially departing from the novel teachings and advantages of the present disclosure. Accordingly, all such modifications, changes, and substitutions are intended to be included within the scope of this disclosure as defined in the following claims. In the claims, any means-plus-function clauses are intended to cover the structures described herein as performing the recited function and not only structural equivalents, but also equivalent structures. Moreover, it is the express intention of the applicant not to invoke 35 U.S.C. § 112(f) for any limitations of any of the claims herein, except for those in which the claim expressly uses the word “means” together with an associated function.

What is claimed is:

1. A method comprising:
introducing an oil comprising metals and an aqueous solution into a microchannel reactor;
wherein the microchannel reactor comprises a hollow conduit having a plurality of fibers disposed therein,
wherein the plurality of fibers form microchannels therebetween; and
wherein the plurality of fibers comprise an enzyme immobilized on surfaces thereof;
enzymatically reacting the oil and the aqueous solution within the microchannels to form a treated oil comprising the oil less at least a portion of the metals and an effluent comprising the aqueous solution and the at least a portion of the metals; and
separating the treated oil and the effluent.

2. The method of claim 1, wherein the enzyme comprises a phospholipase; and
wherein the oil is soybean oil comprising phospholipids and the metals comprise phosphorous contained within the phospholipids.

3. The method of claim 1, wherein, during introducing the oil and the aqueous solution, a total flow rate of the oil and the aqueous solution per cross-sectional area of the hollow conduit is from 20 ml/min/cm² to 250 ml/min/cm².

4. The method of claim 2, wherein introducing the oil and the aqeuous solution comprises:
premixing the oil and the aqueous solution at a weight ratio of from 85:15 to 98:2 and then introducing the mixture into the hollow conduit.

5. The method of claim 2, wherein introducing the oil and the aqueous solution comprises introducing the oil at a first rate and the aqueous solution at a second rate, wherein a ratio of the first rate to the second rate is from 85:15 to 98:2.

6. The method of claim 2, wherein the oil further comprises a first content of free fatty acids and the treated oil comprises a second content of free fatty acid, the second content being greater than the first content.

7. The method of claim 1, further comprising, before introducing the oil and the aqueous solution, pretreating the oil by contacting the oil with a pretreatment solution comprising a chelating acid at a temperature of 40 to 90° C.

8. The method of claim 7, wherein the chelating acid comprises citric acid or phosphoric acid and wherein pretreating further comprises adjusting a pH of the pretreatment solution to between 4 and 6.

9. The method of claim 1, wherein separating the treated oil and the effluent comprises centrifuging a mixture of the treated oil and the effluent.

10. A method comprising:
providing a hollow conduit;
disposing a plurality of fibers within the hollow conduit, wherein the fibers comprise hydroxyl groups on surface thereof;
contacting an anchor group precursor in an ethanolic/water solution with the fibers to bind an anchor group to the hydroxyl groups of the fibers;
contacting an aqueous solution of a bifunctional crosslinker with the anchor group bound to the fibers; and
contacting an aqueous solution of enzyme with the fibers to bind the enzyme to the fibers.

11. The method of claim 10, further comprising:
after binding the enzyme to the fibers, introducing water and a crude oil comprising phospholipids into the hollow conduit;
reacting the water and the crude oil with the enzyme bound to the fibers, wherein said reaction yields a treated oil having at a reduced content of phospholipids and an effluent comprising the water and impurities removed from the crude oil, said impurities comprising at least phosphorous from the phospholipids of the crude oil; and
separating the treated oil and the effluent.

12. The method of claim 11, wherein the enzyme is a phospholipase and reacting the water and the crude oil with the enzyme digests at least a portion of the phospholipids of the crude oil.

13. The method of claim 11, wherein introducing the water and the crude oil comprises:
premixing the crude oil and the water at a weight ratio of from 85:15 to 98:2 and then introducing the mixture into the hollow conduit; or
introducing the crude oil at a first rate and the water at a second rate, wherein a ratio of the first rate to the second rate is from 85:15 to 98:2.

14. The method of claim 11, wherein, during introducing the water and the crude oil, a total flow rate of the water and the crude oil per cross-sectional area of the hollow conduit is from 20 ml/min/cm² to 250 ml/min/cm².

15. The method of claim 10, wherein the anchor group precursor is aminopropyl triethoxy silane or dopamine hydrochloride, the bifunctional crosslinker is glutaraldehyde, and the enzyme is a phospholipase.

16. The method of claim 10, further comprising removing the enzyme from the fibers by simultaneously introducing a strong base and an alcohol into the hollow conduit.

17. The method of claim 16, further comprising reapplying the enzyme by repeating the contacting an anchor group precursor, the contacting an aqueous solution of a bifunctional crosslinker, and the contacting an aqueous solution of enzyme.

18. An apparatus comprising:
a hollow conduit having an inlet, an outlet, and a plurality of fibers disposed therein;
wherein the plurality of fibers form microchannels therebetween;
wherein the plurality of fibers comprise an enzyme immobilized on surfaces thereof; and
wherein the enzyme is immobilized on the plurality of fibers via a silane compound.

19. The apparatus of claim 18, further comprising a mixing tank comprising a mixing implement, the mixing tank being fluidically coupled to the inlet.

20. The apparatus of claim 18, wherein the silane compound is an amino silane or polydopamine, the enzyme is a phospholipase, and the enzyme is covalently bound to the silane compound via glutaraldehyde.
